(19) United States
(12) Patent Application Publication
(10) Pub. No.: US 2013/0236955 A1
(43) Pub. Date: Sep. 12, 2013

(54) CELL PASSAGE DEVICE

(71) Applicant: Roland Huchler, Stuttgart (DE)

(72) Inventor: Roland Huchler, Stuttgart (DE)

(21) Appl. No.: 13/988,174

(22) PCT Filed: Nov. 17, 2010
(86) PCT No.: PCT/EP2010/006995

§ 371 Date: May 17, 2013

(51) Int. Cl. C12N 5/00

(52) U.S. Cl. CPC C12N 5/00
USPC 435/325; 435/283.1

(57) ABSTRACT

A cell passage device for automatic passage of a suspension of biological tissue or cells from a first culture vessel into one or more additional culture vessels includes a sampling unit, a dosing unit, at least one reservoir for fluid, a pressure recipient and a fluid block. The sampling unit removes the suspension from the first culture vessel and includes at least one ascending tube and at least one pressure tube. An ascending tube receives the suspension. A pressure tube pressurizes the first culture vessel. The ascending tube and pressure tube are inserted into the first cell culture vessel and sealable with the first cell culture vessel in a pressure-proof manner. The dosing unit doses the suspension into additional culture vessel(s). The fluid block has horizontally oriented fluid channels and valves assigned them, making fluid connections with the sampling unit, dosing unit, at least one reservoir and the pressure recipient.

The present invention is in the field of automatic culturing and further processing of biological cells and tissues and relates to a device and a method for automatic passage of biological cells or tissues in cell culturing via pressurization.

In the production of cells and tissues or tissue layers composed of cells, measures for cell proliferation and/or cell reproduction constitute central process steps. Biological cells are capable of division; cell complexes and optionally even entire tissue layers can develop over a certain culturing period from individual cells. This process is repeated iteratively for effective cell reproduction. Individual cells, for example, are cultured to form a tissue complex and then the tissue complex is dissolved and the cells are separated. The separated cells are then cultured again to each form tissue or cell complexes. This process is referred to as a “passage.” The passage of cells requires in particular the transfer of cells from a first cell culture vessel to one or more other cell culture vessels. This takes place in known manual processes by preparing the most homogeneous possible cell suspension in the first culture vessel, removing the cell suspension by means of a pipette with suction and aliquoting the cell suspension drawn up into the pipette into the additional cell culture vessels. Known approaches for solving the problems of automated passage have been based on a simulation of these manual processes using technical means. To do so, ultimately the arm and the hand of the laboratory personnel are replaced by suitable robotic kinematics. The traditional manual passage sequence will now be described on the example of the passage of adherent cells. In a first step, the culture vessel containing the adherent cells is opened and then in a second step the supernatant above the cells, usually consisting of spent culture medium, is removed by suction, and then in another step, the cells adhering to the bottom of the culture vessel are optionally rinsed by overlayering them several times with buffer solution. In another step, an enzyme solution (trypsin) is added to break up the tissue complex and to release the cells from the bottom of the vessel, and the cells are usually incubated with agitation. In an additional step, the enzyme activity is stopped by an enzyme inhibitor to be added. In another step, the cells and cell complexes or tissue fragments that have been released are suspended in a culture medium. In another step, the suspension is removed from the culture vessel with suction. In an optional step, the cell density (cell count per unit of volume) in the suspension is determined. In the final steps, the cell suspension is distributed (aliquoted) to other cell culture vessels and the cell culture vessels are the sealed.

Very high demands are made of this process. All handling steps must be performed under sterile conditions and any contamination of the cell material must be prevented. The liquid quantities are to be dosed with a precision that goes down to a few volume percent because otherwise the reproducibility and the investigations based on the cell cultures can be falsified. In conjunction with the passage of adherent cells, it is necessary to use enzymatic processes. A predefined dwell time must be maintained here exactly. At the same time, an automated system is required to be flexible in use to be able to use different cells and different culturing approaches. The adaptability of the volumes, the adaptations of different culture media and reagents, especially enzymatic solutions, dye solutions and auxiliary fluids are also required. Since the passage and media replacement must be performed regularly and repeated, the time required for this should be minimal. It is also required that the effort for the required infrastructure, especially the equipment complexity for implementation of automated passage must be as low as possible.

The known automation of the known manual process requires a high equipment complexity to simulate the working steps and actions taken by the laboratory personnel. At the same time the known automation does not overcome the essential disadvantages of manual passage. These disadvantages include mainly the risk of contamination, uncontrolled mechanical stress on the biological cells during the pipetting operation, which is characterized by the uncontrollable flow conditions due mainly to alternating application of a vacuum and excess pressure (suction, pumping) and repeated changes in the direction of flow. In addition, inaccuracies occur in aliquoting the cells because of separation of during the dwell time of the suspension in the pipette.

The present invention is based on the technical problem of supplying a device for automatic passage of biological tissue or cells (cell passage device) which will largely or completely overcome the disadvantages of the state of the art as described above. The present invention solves this technical problem in particular by completely departing from the principles of the solutions known from manual methods of passage. The use of a pipette in particular is avoided.

According to a first aspect, the invention relates to a cell passage device for automatic passage of a suspension of biological tissue or cells or fluid from a first culture vessel into at least one or more additional culture vessels. According to the present invention, this device contains at least one sampling unit for receiving and removing the suspension or fluid from the first culture vessel. The sampling unit has at least one ascending tube to receive the suspension or fluid and at least one pressure tube for pressurizing the first culture vessel. It is provided in particular that the ascending tube and the pressure tube of the sampling unit can be inserted into the first cell culture vessel, and the sampling unit can seal the culture vessel in a pressure-proof manner. The sampling unit is designed in particular so that the ascending tube in the inserted state is immersed in the suspension or fluid in the first cell culture vessel, and the pressure tube is above the fluid level.

In addition, the cell passage device according to the invention has at least one dosing unit for dosing the suspension or fluid into one or more additional culture vessels. The dosing unit is specially designed to transfer the suspension or fluid sampled by the integral sampling unit to one or more of the additional culture vessels, in particular to aliquot said suspension or fluid so that it is distributed to several additional culture vessels.

Furthermore, the cell passage device according to the invention has at least one reservoir for fluid (fluid reservoir). Multiple fluid reservoirs are preferably provided. The fluid reservoirs are specially designed to receive the media required for the cell passage process such as cell culture medium (nutrient medium), buffer or washing fluid, enzyme solution and optionally resuspension fluid as well as sterilization fluid. The amount required for the passage process can be dosed automatically from the reservoirs.

In addition, the cell passage device according to the invention has a pressure recipient which is specially designed to supply the excess pressure required for performing the passage process according to the invention. The pressure recipient is preferably optionally connectable to one or more of the aforementioned elements to permit fluid transport (transport of suspension) along the pressure gradient thereby formed optionally against the force of gravity (hydrostatic pressure).

Another element of the cell passage device according to the present invention is a fluid block which has a plurality of fluid channels and a plurality of valves assigned to the fluid channels. The fluid channels are in fluid connection with the elements described above, in particular with the sampling unit, dosing unit, at least one fluid reservoir and the pressure recipient. The fluid block, fluid channels and valves here are specially designed to selectively establish a fluid connection between one or more of the preceding elements and at least one additional one of the preceding elements to perform the passage process, preferably sequentially to permit a selective pressurization and thereby permit a targeted fluid transport within the cell passage device.

The fluid block is preferably further characterized in that the fluid channels in the fluid block are oriented essentially horizontally. According to this invention this is to be understood to mean that the fluid channels are oriented essentially at a right angle to the vector for the force of gravity. The cell passage device according to the invention is specially designed to minimize the dwell time of a suspension in fluid lines running essentially parallel to the gravity vector. The influence of the gravity-induced separation of the suspension on the distribution of the suspended cells or tissue can be prevented or reduced.

The invention provides that through pressurization of the first culture vessel by means of the at least one pressure tube of the integral sampling element, the fluid or suspension in the first cell culture vessel can be removed through the at least one ascending tube of the integral sampling unit.

Biological cells or tissue parts present in the suspension can thus be gently removed from the first cell culture vessel through the pressurization according to the invention in a controlled manner and transferred to the dosing unit by way of the fluid block which may preferably also serve as temporary storage for the suspension. By pressurizing the suspension thereby removed, the suspension can be transferred through the dosing unit into one or more additional culture vessels.

The invention also provides that the media stored in the fluid reservoirs are dosed sequentially and selectively through pressurization of the fluid reservoir into the first culture vessel via the fluid block and the integral sampling unit. In a special embodiment, the dosing of enzyme solution into the first culture vessel is thus made possible to bring adherent cells there into suspension to remove them from the culture vessel.

The approach according to the invention is based primarily on the fact that fluid or suspension is not removed from the culture vessel by suction, stored temporarily in a pipette and dispensed by dosing it. Instead, the fluid or suspension is forced by pressurization through the pressure tube according to the invention of the integral sampling unit into an ascending tube of the sampling unit which is immersed in the fluid or suspension according to the invention and optionally removed optionally without further temporary storage and while retaining the direction of flow. In this way, the suspension can be removed from the first culture vessel in particular and “forced” into the downstream dosing unit for dosing the suspension into one or more additional culture vessels in one operation while retaining the direction of flow, preferably without temporary storage in between. This avoids any change between vacuum and excess pressure, temporary storage with the dwell times and the risk of separation associated with that and the multiple reversals of the direction of flow, which have a negative effect.

For conveyance and dosing of fluid or suspension, the present invention also preferably provides that the amount of fluid is controlled by the opening time of the valves in the fluid block, taking into account the specific velocity of flow, at an essentially constant pressure. The fluid block together with the valves functions as a central control unit to enable the flow of fluid and suspension in the device. The valves are preferably triggered in a program-controlled process. In a special embodiment, the program control allows an adaptation of the dosing amounts, a predetermination of the number of aliquots in combination with the number of additional culture vessels provided as well as preselection of additional washing, sterilization and/or calibration steps.

In a preferred embodiment of the sampling unit, the ascending tube and pressure tube together are designed as an integral unit in the form of a double needle or multiple needles. This needle is preferably inserted through an elastic septum which is formed on the culture vessel so that the septum preferably seals the culture vessel in a pressure-proof manner in the area where the needle punctures through it. In another embodiment, additional means are provided on the sampling unit for pressure-proof sealing of the culture vessel. These means may be designed as sealing lips, flat gaskets or labyrinth gaskets in a known manner such that in a special embodiment these gaskets engage with the culture vessel to form a seal.

Due to the use of excess pressure according to the invention, the risk of contamination in the system as a whole can be prevented. The excess pressure is expediently 0.5 to approximately 2 bar. The gas required for pressurization may be supplied in a sterile form which is free of contamination. In a first variant the gas is ambient air. In addition the system allows the use of gases or gas compositions which are favorable for cell culturing for example oxygen gas enriched with carbon dioxide (95% O₂, 5% CO₂). This allows the use of physical buffer systems which are gentle to the cells in the cell culture medium. In the simplest case the pressure may be supplied via a connected gas bottle. A mechanical pump is then superfluous, which further reduces the risk of contamination and simplifies maintenance.

In a special embodiment, the cell passage device according to the invention makes it possible to perform the passage on the basis of a purely unidirectional flow of the suspension. This makes it possible to reduce the risk of formation of residues which exists in known pipetting methods due to the flow reversal, which is required there. In a special embodiment, the device according to the invention allows an essentially continuous transport/flow of suspension in the passage so that the dwell time of the suspension within the sample unit can be minimized in particular. Therefore the sampling unit may optionally be guided in parallel with the gravity vector (vertically). Retention of the suspension in the device according to the invention, which may optionally be necessary, then occurs only in the area of the horizontal fluid channels in the fluid block. Based on the horizontal arrangement of the fluid channel according to the invention, essentially perpendicular to the gravity vector, the separation of the suspension due to gravity, i.e., an unwanted sedimentation of the cells in the fluid channel, is prevented.

In a special embodiment, the device according to the invention prevents any kinematic processes from taking place within the system formed by the sampling unit, the dosing unit, the fluid reservoir, the pressure recipient and the fluid blocks. Therefore dead volumes can be minimized advantageously. In addition, kinematic processes, for example, piston strokes are also subject to wear. According to the invention, this makes it possible to rule out the risk of loss of a seal due to wear and contamination due to abrasion on moving parts within the system.

Media that may be used to implement the passage include in particular buffer solutions or culture media enriched with nutrient substances which are known per se. For the separation of cells and release of adherent cells to prepare a suspension, enzyme solutions in particular trypsin may be used, optionally in combination with EDTA. Additional essentially proteolytic enzymes are also very suitable for separation of cells and in preparation for the passage. In addition, cleaning media for the lines, vessels and valves may be supplied by means of a fluid reservoir according to the invention in order to remove cells and other contaminants from the entire device, for example, in preparation for a passage process or following such a process. Suitable cleaning media include in particular ethanol, propanol, peracetic acid and/or hydrogen peroxide.

To permit reliable transport of fluid and suspension by pressurization by means of air or gas, it is provided in particular that the cross section of the fluid channels must not exceed a certain size depending on the physical properties of the fluids and the wall material of the fluid channels, in order to ensure reliable formation of the meniscus and to prevent mixing of air or gas with fluid or suspension. In addition an abrupt change in cross section of the fluid channels, i.e., especially abrupt transition, corners and edges are prevented in a special embodiment in order to prevent cavities and the formation of a vacuum with the risk of outgassing of the fluid or suspension during flow. Foaming in the system can therefore be prevented.

Another aspect of the present invention is a method for automatic passage of cells or tissues in suspension from a first culture vessel into one or more additional culture vessels using the cell passage device according to the invention as described herein. This method has at least the following steps according to the present invention. In a first step, the sampling unit containing at least one ascending tube and at least one pressure tube is inserted into the first culture vessel, and the culture vessel is sealed in a pressure-proof manner, preferably via the sampling unit. In another step, the interior of the culture vessel is pressurized via the pressure tube, so that fluid or suspension in the culture vessel is removed through the at least one ascending tube, following the pressure gradient.

In a special embodiment, the method according to the invention is further characterized in that the fluid or suspension removed through the ascending tube is transferred via a dosing unit which is connected to the ascending tube to at least one additional culture vessel.

The pressurization of the culture vessel via the pressure tube is preferably the only driving force for removing the fluid or suspension from the culture vessel and preferably for transferring the fluid or suspension through the dosing unit to at least one further culture vessel. This method further preferably provides that for transferring the fluid or suspension out of the first culture vessel into the at least one additional culture vessel, a fluid connection, at least temporary, between the ascending tube and the dosing unit is established. This fluid connection preferably has fluid flowing through it always in the same direction of flow during the automatic passage of the fluid or suspension.

The present invention will now be described in greater detail on the basis of the following figures and concrete exemplary embodiments although these are not to be understood as restrictive in any way.

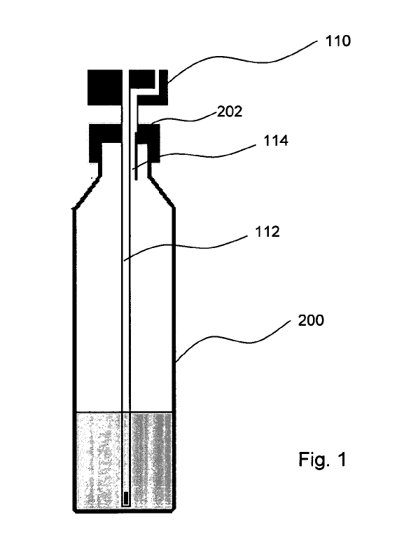

FIG. 1 shows an embodiment of the sampling unit (110) according to the invention having an ascending tube (112) to receive the suspension or another fluid and a pressure tube (114) for pressurization, inserted into a culture vessel (200). In the embodiment shown here the ascending tube (112) and the pressure tube (114) are designed as an integral double needle which is inserted in the area of the septum (202) of the first culture vessel (200). The culture vessel (200) is thus sealed in a pressure-proof manner with respect to the environment.

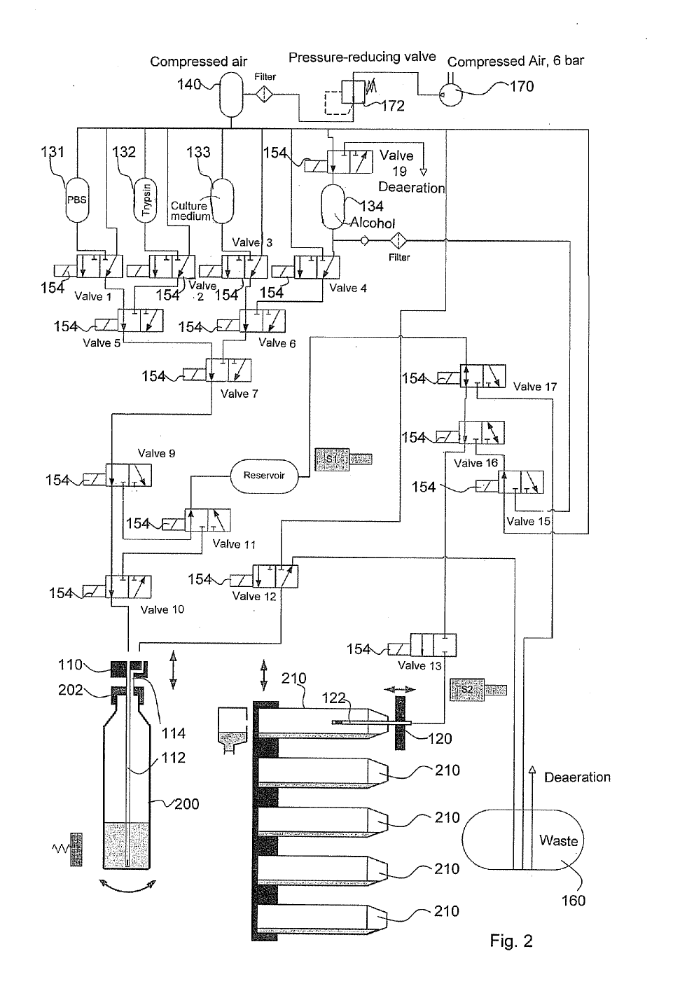

FIG. 2 shows overall design of a special embodiment of the cell passage device (100) according to the invention having a sampling unit (110), a dosing unit (120), fluid reservoirs (131, 132, 133, 134) and a pressure recipient (140). The reservoir (131) preferably contains buffer solution, the reservoir (132) preferably contains enzyme solution, the reservoir (133) preferably contains culture medium, the reservoir (134) preferably contains cleaning fluid. The dosing unit (120) has a single needle (122), which can be selectively introduced into the additional culture vessel (210) to dose the suspension or fluid there. The valves (154) and the fluid connections between the valves and the aforementioned elements are arranged in an integral fluid block (150) (not shown in the schematic diagram). In the embodiment shown here, the valves are electromagnetically actuated. The control is by means of a central computer using a program implemented in the hardware/software. A waste container (160) and a pressure pump (170) with a pressure-reducing valve (172) are optionally provided.

### EXAMPLE

### Automated Passage

The first culture vessel (200) containing the cells or tissue to be subjected to the passage is made available. The sampling unit (110) with the ascending tube (112) and the pressure tube (114) is introduced into the culture vessel (200), such that the ascending tube (112) is positioned beneath the fluid level, as close to the bottom of the culture vessel (200) as possible. The pressure tube (114) is above the fluid level in the culture vessel. By switching at least one valve (154) in the fluid block (150), the pressure recipient (140) is connected to the pressure tube (114) to apply an excess pressure to the interior of the culture vessel (200). As a result of this excess pressure, the fluid in the culture vessel (200) escapes through the ascending tube (112) and is thus conveyed out of the culture vessel (200) in the direction of the fluid block (150). In this initial fluid transfer step, the supernatant culture medium is removed from the culture vessel (200). The culture medium removed from the culture vessel (200) by way of the fluid block (150) is removed from the system. The ascending tube (112) is preferably connected to an optional waste container (160) via the fluid blocks (150) and the culture medium is removed from the culture vessel (200) through the ascending tube (112) and the fluid block (150) into the optional waste container (160) by means of the pressurization of the culture vessel (200).

In another step, buffer solution is added for washing the cells or tissue remaining in the culture vessel (200). To do so, the fluid reservoir containing buffer solution (131) is connected to the pressure recipient (140) by switching at least one valve within the fluid block (150) in order to dose buffer solution from the reservoir (131). The buffer solution dosed from the reservoir (131) is dosed to the culture vessel via the ascending tube (112) or optionally via a further supply tube of the sampling unit (113, not shown), preferably by switching at least one additional valve within the fluid block (150). Next, the added buffer solution is removed through the ascending tube (112) above by pressurizing the culture vessel (200) via the pressure tube (114) as described above and preferably transferred to the optional waste container (160). This washing procedure is optionally repeated one or more times.

According to the addition of buffer solution from the reservoir (131) as described above, enzyme solution from the reservoir (132) is then removed through the ascending tube (112) or optionally through the additional supply tube (113) into the culture vessel (200) for releasing adherent cells in preparation for suspension by actuating the corresponding valves in the fluid block (150) or is optionally removed to the culture vessel (200) via the additional supply tube (113) in order to incubate the cells with enzyme solution in the culture vessel (200). After the end of the incubation time and optionally through the support of the separation process by mechanical vibrations (ultrasound, etc.), this process is stopped by adding a solution that inhibits the enzyme activity (similar to the addition of a buffer solution, as described above). To do so, the culture medium from the reservoir (133) in particular is dosed to the culture vessel (200) by actuation of at least one corresponding valve in the fluid block (150) via the ascending tube (112) or the optional additional supply tube (113). The cells thus released are suspended (resuspended) in the culture medium thereby dosed. The formation of a largely homogeneous suspension is optionally supported by additional mechanical measures (ultrasound, etc.) in the culture vessel (200).

As in the steps described above, the suspension thus obtained is sent to the fluid block (150) via the ascending tube (112) and then ultimately sent to the dosing unit (120) via the fluid block (150) by pressurizing the culture vessel (200) via the pressure tube (114). Dosing of the suspension to the dosing unit (120) and thus aliquoting of the suspension among the plurality of culture vessels (210) are thus made possible preferably through targeted control of the opening times of the valves.

In an optional further step after successful passage, the device is flushed and cleaned by rinsing with cleaning fluid from the reservoir (134) to prevent cross-contamination between individual passages.

1-5. (canceled)

6. A cell passage device for automatic passage of a suspension of biological tissue or cells from a first culture vessel into one or more additional culture vessels, the cell passage device comprising:
sampling unit for removing the suspension from the first culture vessel, the sampling unit including at least one ascending tube and at least one pressure tube, the at least one ascending tube for receiving the suspension, the at least one pressure tube for pressurizing the first culture vessel, the ascending tube and pressure tube insertable into the first cell culture vessel and sealable with the first cell culture vessel in a pressure-proof manner;
a dosing unit for dosing the suspension into one or more of the additional culture vessels;
at least one reservoir for fluid;
a pressure recipient; and
a fluid block having a plurality of fluid channels and valves assigned to the fluid channels such that the fluid channels are in fluid connection to the sampling unit, dosing unit, at least one reservoir and the pressure recipient, the fluid channels are oriented horizontally in the fluid block.

7. A method of automatic passage of a suspension of a biological tissue or cells from a first culture vessel into one or more additional culture vessels with the cell passage device of claim 6, the method comprising:
inserting the sampling unit into the first culture vessel and sealing the first culture vessel in a pressure-proof seal; and
pressurizing an the interior of the first culture vessel via the pressure tube so that suspension in the first culture vessel is removed through the ascending tube.

8. A method of automatic passage of a suspension of biological tissue or cells from a first culture vessel into one or more additional culture vessels, the method comprising:
inserting a sampling unit having an ascending tube and a pressure tube into the first culture vessel and sealing the first culture vessel in a pressure-proof seal; and
pressurizing an the interior of the first culture vessel via the pressure tube so that suspension in the first culture vessel is removed through the ascending tube.

9. The method according to claim 8, further comprising transferring the suspension removed through the ascending tube to at least one additional culture vessel via a dosing unit.

10. The method according claim 8, wherein the pressurization of the anterior of the first culture vessel via the pressure tube is the only driving force for removing the suspension from the first culture vessel.

11. The method according to claim 8, wherein a fluid connection between the ascending tube and the dosing unit is established for transferring the suspension from the first culture vessel into the at least one additional culture vessel, so that the suspension always flows through the fluid connection in the same direction of flow.
